(19) United States
(12) Patent Application Publication
(10) Pub. No.: US 2019/0366015 A1
(43) Pub. Date: Dec. 5, 2019

(54) DISCRETE VAPORIZER EXHALATION

(71) Applicant: INDOSE INC, Woodland Hills, CA (US)

(72) Inventor: Daniel Freeman, Woodland Hills, CA (US)

(73) Assignee: INDOSE INC, Woodland Hills, CA (US)

(21) Appl. No.: 16/426,101

(22) Filed: May 30, 2019

Related U.S. Application Data
(60) Provisional application No. 62/679,048, filed on Jun. 1, 2018.

(51) Int. Cl. A61M 11/04; A61M 16/18

(52) U.S. Cl. CPC A61M 11/04; A61M 16/18

(57) ABSTRACT

A vaporizer includes a mouthpiece that is configured to permit a user to inhale a vaporized substance from the vaporizer, and permit the user to exhale vapor into the vaporizer. The vaporizer includes an inhalation pathway that is configured to deliver the vaporized substance to the mouthpiece to permit the user to inhale the vaporized substance from the vaporizer through the mouthpiece. The vaporizer includes an exhalation pathway that is configured to receive exhaled vapor that is exhaled by the user into the vaporizer through the mouthpiece and at least substantially remove the exhaled vapor from exhausted air.

### CROSS-REFERENCE TO RELATED APPLICATION

This application claims priority under 35 U.S.C. § 119 to U.S. Application No. 62/679,048 filed on Jun. 1, 2018, in the United States Patent & Trademark Office, the disclosure of which is incorporated herein by reference in its entirety.

### BACKGROUND

Vaporizers are used to vaporize a substance, generally a drug, to give the user the ability to inhale the drug. Generally, the vaporized substance is stored in liquid form in a storage area. An atomizer then changes the state of the liquid into a gas or vapor or aerosol, as to be suspended in air. The user then inhales this substance. One of the advantages of vaporizers is that the substance is quickly delivered to the user's lungs and blood stream. This process is considered healthier than traditional smoking methods that combust the substance. After inhalation, the user exhales the vapor into the surrounding environment.

After use and upon exhalation, the vapor lingers around for some time. The exhaled vapor cloud expands and eventually dissipates into the surroundings. The vapor cloud is visible and has an odor. There are many situations in which a user may not want to see a visible cloud of vapor, nor smell the vapor, nor be exposed to the particles in the vapor.

For example, if a user uses a vaporizer indoors, the visible vapor may pose an annoyance to themselves and others around. Beyond a visual nuisance, others may not want to be exposed to nor inhale the vapor. The smell itself may be a nuisance. Exhaled vapor often contains some amount of the drug/vaporized substance. When the user exhales into the environment, others in the area will likely inhale some of that drug/substance and be exposed to its effects. Many find this undesirable or unsafe.

### SUMMARY

According to an aspect of the disclosure, a vaporizer includes a mouthpiece that is configured to permit a user to inhale a vaporized substance from the vaporizer, and permit the user to exhale vapor into the vaporizer; an inhalation pathway that is configured to deliver the vaporized substance to the mouthpiece to permit the user to inhale the vaporized substance from the vaporizer through the mouthpiece; and an exhalation pathway that is configured to receive exhaled vapor that is exhaled by the user into the vaporizer through the mouthpiece.

### BRIEF DESCRIPTION OF THE DRAWINGS

FIG. 1 is a diagram of an example of a vaporizer according to an embodiment of the disclosure;

FIG. 2 is a diagram of an example exhalation pathway according to an embodiment of the disclosure;

FIG. 3 is a diagram of an example exhalation pathway according to an embodiment of the disclosure; and

FIG. 4 is a diagram of an example exhalation pathway according to an embodiment of the disclosure.

### DETAILED DESCRIPTION

This disclosure provides a way of trapping vapor particles upon exhalation. In one embodiment, the user can exhale into the vaporizer, using the same mouthpiece as was used when inhalation occurred. The exhaled vapor travels through the vaporizer in such a way as to trap the vapor. The vapor would travel through a filter designed to capture the vapor with minimal impact on user experience.

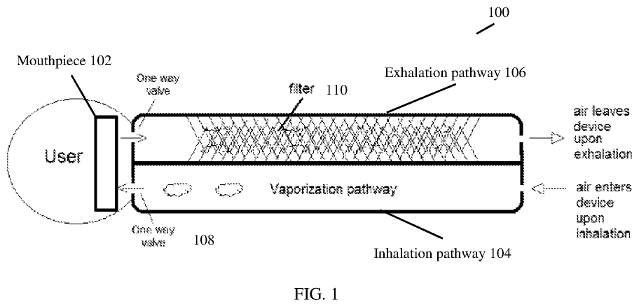

FIG. 1 is a diagram of an example vaporizer according to an embodiment of the disclosure. As shown in FIG. 1, a vaporizer 100 may include a mouthpiece 102, an inhalation pathway 104, an exhalation pathway 106 and a valve 108. The vaporizer 100 may include other components that are not shown in FIG. 1, such as, for example, an atomizer, a reservoir for the inhalable substance, a processor, and/or the like, as is known in the art, for example as in application Ser. No. 15/244,518 of the present applicant, which is incorporated herein by reference.

The vaporizer 100 may be used to vaporize a substance for a user to inhale through the mouthpiece 102.

The mouthpiece 102 may be arranged at one end of the vaporizer 100 for the user to inhale and exhale the vaporized substance. However, the mouthpiece 102 may be arranged anywhere else on the vaporizer 100. According to an embodiment, the mouthpiece 102 may be arranged so that a part of the mouthpiece 102 is connected to the inhalation pathway 104 and the other part of the mouthpiece 102 is connected to the exhalation pathway 106. In another embodiment, the mouthpiece 102 may be connected to both the inhalation pathway 104 and the exhalation pathway 106 for both inhalation and exhalation by the user.

The vaporizer 100 may include the inhalation pathway 104 configured to move or carry the vaporized substance to the mouthpiece 102. The inhalation pathway 104 may have at least one opening that allows air to enter the inhalation pathway upon the user inhaling the vaporized substance. The size of the opening may vary and may be predetermined or determined based on the user setting. The inhalation pathway 104 may include a one-way valve that may open or close based on the user's input. For example, the user may press a button disposed on the vaporizer 100 to open the one-way valve in the inhalation pathway 104 and release the button to close the one-way valve disposed in the inhalation pathway 104. The one-way valve may be arranged so that it is disposed on the opposite end of the vaporizer from the opening. However, the arrangement is not limited hereto. The one-way valve may alternatively be of a type which will open in response to the user's inhalation, and close upon exhalation.

The vaporizer 100 may include the exhalation pathway 106 that receives, through the mouthpiece 102, the exhaled vapor by the user. The exhalation pathway 106 is configured to reduce vapor cloud generated upon the user's exhalation of the vaporized substance. The exhalation pathway 106 may include its own one-way valve configured to receive the exhaled vapor by the user. The one-way valve may be configured within the vaporizer 100 in such a way as to allow airflow only in the desired fashion, e.g., during exhalation. The exhalation pathway 106 may also include an opening configured to dispose of or exhaust air upon exhalation. Notably, the exhalation pathway 106 may include a filter to trap or remove vapor particles upon exhalation. The user may exhale the vapor into the vaporizer 100 using the same mouthpiece as the inhalation. The exhaled vapor travels through the exhalation pathway 106 so as to trap the vapor. In other words, the exhaled vapor may be trapped in the exhalation pathway 106 to reduce vapor cloud exhausted to the surroundings. According to another embodiment, the exhalation pathway 106 may be entirely separated from the vaporizer device 100. In such embodiment, the device could be a standalone product or an add-on product. As such, the user may use a vaporizer of his or her own choice for inhalation and then exhale into the standalone device.

According to an embodiment, the vaporizer 100 may include at least one valve 108. For example, a one-way valve may be disposed in the inhalation pathway 104 and another one-way valve may be disposed in the exhalation pathway 106. The valve may be configured so that it may open or close based on the user's input to the vaporizer 100. Also, the valve may be configured to partially open based on the user's input.

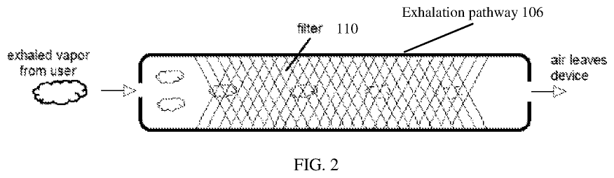

FIG. 2 is a diagram of an example exhalation pathway according to an embodiment of the disclosure. As shown in FIG. 2, an exhalation pathway 106 may include a filter 110.

The filter 110 may be configured to capture or trap the vapor particles exhaled by the user. However, the disclosure is not limited to use of the filter to capture the vapor. A pathway may be designed to bias or facilitate deposition or re-condensation of the vapor in the pathway. The filter 110 may substantially reduce the exhaled vapor in the surrounding area by trapping and/or condensing the exhaled vapor from the user. However, it should be noted that a total elimination of the vapor may not be necessary.

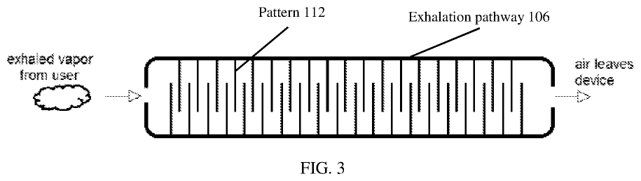

FIG. 3 is a diagram of an example exhalation pathway according to an embodiment of the disclosure. As shown in FIG. 3, an exhalation pathway 106 may include a pattern 112 formed by placing obstructions or walls so that the vapor exhaled by the user travels an elongated path in a twist-turn or serpentine manner.

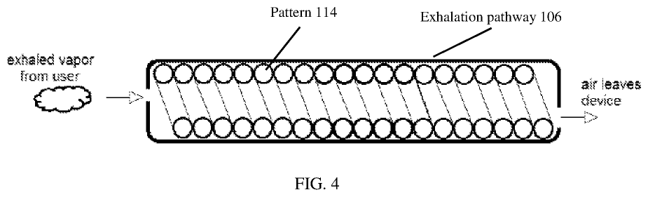

FIG. 4 is a diagram of an example exhalation pathway according to an embodiment of the disclosure. As shown in FIG. 4, an exhalation pathway 106 may include a pattern 114 formed by placement of a tube or the like so that the exhaled vapor may travel through the tube in a spinning manner. The tube may be in the form of a coil or the like, with either being of constant or variable diameter.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A vaporizer, comprising:
a mouthpiece that is configured to permit a user to inhale a vaporized substance from the vaporizer, and permit the user to exhale vapor into the vaporizer;
an inhalation pathway that is configured to deliver the vaporized substance to the mouthpiece to permit the user to inhale the vaporized substance from the vaporizer through the mouthpiece; and
an exhalation pathway that is configured to receive and deliver exhaled vapor that is exhaled by the user into the vaporizer through the mouthpiece.

2. The vaporizer of claim 1, wherein the exhalation pathway is, at least, partially separated from the inhalation pathway within the vaporizer.

3. The vaporizer of claim 1, further comprising:
a channel for the inhalation pathway comprising at least one opening configured to receive air; and
a one-way valve for the inhalation pathway configured to receive and pass the vaporized substance to the user.

4. The vaporizer of claim 1, further comprising:
a channel for the exhalation pathway comprising at least one opening configured to deliver air and the exhaled vapor that is exhaled by the user; and
a one-way valve for the exhalation pathway configured to receive and pass the exhaled vapor that is exhaled by the user.

5. The vaporizer of claim 4, wherein the exhalation pathway further comprises a filter disposed in the channel for the exhalation pathway configured to remove or trap vapor particles of the exhaled vapor.

6. The vaporizer of claim 4, wherein the exhalation pathway is arranged with walls so that the exhaled vapor travels in a twist and turn or serpentine manner.

7. The vaporizer of claim 4, wherein the exhalation pathway is arranged with a tube so that the exhaled vapor travels in a spinning manner.

8. The vaporizer of claim 1, wherein the exhalation pathway is arranged to bias deposition of the exhaled vapor.

9. The vaporizer of claim 1, wherein the exhalation pathway is designed to re-condense the exhaled vapor.

10. A device, comprising:
a mouthpiece configured to permit a user to exhale vapor;
an exhalation pathway that is configured to receive and deliver exhaled vapor that is exhaled by the user; and
a filter configured to remove or trap vapor particles of the exhaled vapor.
